(19) United States
(12) Patent Application Publication
(10) Pub. No.: US 2011/0251233 A1
(43) Pub. Date: Oct. 13, 2011

(54) FORMULATION FOR THE BUCCAL TRANSMUCOSAL ADMINISTRATION OF SETRONS

(76) Inventors: Philippe Perovitch, Le Temple (FR); Marc Maury, Saint Medard En Jalles (FR)

(21) Appl. No.: 13/140,745

(22) PCT Filed: Dec. 17, 2009
(86) PCT No.: PCT/FR2009/052590

§ 371 Date: Jun. 17, 2011

(30) Foreign Application Priority Data
Dec. 19, 2008 (FR) 0807258

(51) Int. Cl. A61K 31/439; A61P 1/08; A61P 1/06; A61K 31/4178

(52) U.S. Cl. 514/299; 514/397

(57) ABSTRACT

The invention provides a formulation for transmucosal administration of at least one active ingredient from the setron family, the formulation comprising said active ingredient in base form and/or in salt form, a hydroalcoholic solution titrating at least 30° alcohol, and optionally a pH correcting agent, said active principle being present in the state of stable and complete dissolution in the hydroalcoholic solution. The invention also provides a method of preparing this formulation and its use for the treatment and prevention of major nausea and/or vomiting syndromes, and also for the treatment and prevention of digestive spasms.

The present invention relates to a formulation for instantaneous buccal transmucosal systemic administration of at least one active ingredient belonging to the setron family.

The invention also relates to a method of the preparation of this formulation and to its use for the treatment and prevention of major nausea and/or vomiting syndromes, as well as for the treatment and prevention of the problem of disabling spasms of the digestive tract.

Setrons are pharmaceutical active ingredients used mainly for the prevention or treatment of nausea and vomiting syndromes linked to cancer therapies. These are powerful lipophilic anti-emetic molecules of low molecular weight, which operate at central nervous system level as receptor antagonists at the 5-hydroxytryptamine-3 (5-HT3) receptor, a subtype of serotonin receptor.

The best known setrons are ondansetron, tropisetron, and granisetron marketed under the respective trade names Zophren®, Navoban®, and Kytril®. There also exist other, less well-known, molecules such as dolasetron and itasetron, which have similar indications, and alosetron, azasetron, benesetron, cliansetron, ramosetron, and zatosetron, having indications in their Notices of Compliance that target treatment of irritable bowel syndrome.

Setrons have a proven anti-emetic activity, but their administration to prevent or treat a major nausea and/or vomiting syndrome linked to the administration of cancer chemotherapy or physical treatment encounters numerous problems.

The quickest and most effective way to administer setrons is by intravenous transfusion. However, this mode of administration requires dedicated personnel and the use of specialized equipment. It is costly and its use is burdensome for the patient, who is already undergoing very many transfusion treatments, in particular for the administration of chemotherapy.

To protect the veins of patients, to facilitate taking the medication, and to reduce costs, it is therefore preferable to avoid the intravenous route and to administer setrons orally.

The best known form of oral administration is enteral administration by means of pills, but this mode of administration is not suitable for administering setrons.

The target patients are extremely sensitive to taking any medication orally and often reject them instantly. Thus anti-emetics administered orally may in themselves induce the syndrome that they are intended to combat.

Apart from this problem, if the setrons administered are ingested properly, the delay before they begin to act is in the range one to three hours from taking them, which delay is out of all proportion to the expectations of a suffering patient.

When they are introduced into the digestive tract and the stomach, lipophilic setron molecules suffer the so-called “digestive first pass” effect, referring to deterioration and losses linked to the environment of the stomach or to variations in intestinal physiology. They are then subject to a so-called “hepatic first pass” effect which leads to them being metabolized and/or degraded more or less intensely, with the formation of numerous metabolites, for the most part inactive or toxic and producing side effects.

The dose of active ingredients that is genuinely bioavailable is therefore low: only a residual part that, in the best possible circumstances, does not exceed 60% of the quantity administered, is in fact distributed to the central nervous system and reaches the 5-HT3 receptors in the brain to produce the expected pharmacological effect.

Thus several major problems are apparent.

The first problem is that a composition must be absorbed by a patient who is already weakened, laid low by serious nausea reflexes. The medication must not be rejected once swallowed and the active ingredient must be sufficiently absorbed despite the digestive problems of the patient.

A second problem is administering a sufficient dose of setrons to the patient, given the weight of the person, and the dilution and dispersion of the active ingredient in the organism, so that the significantly active part that actually reaches the 5-HT3 receptors in the brain proves effective.

Another problem is the latency time caused by metabolization and diffusion in the organism before the setron molecule acts and the patient experiences the benefits thereof.

Administration of setrons via the digestive tract is therefore not appropriate.

Other possible ways of administering setrons are known, such as transcutaneous administration, which is generally effected with the aid of gel-type semi-solid systems or reservoir-type solid systems. For example, application US-2007/0225379 describes in its examples J and K gels based on granisetron or ondansetron and their administration via the skin. Those are complex systems, however, intended for long-term administration, which therefore do not allow instantaneous passage into the blood of a therapeutic dose of the active ingredient, therefore making them incompatible with the immediate treatment of a nausea and/or vomiting syndrome.

Finally, there is also the per/sublingual route allowing medications to be administered by passive passage through the mucosa under the tongue, of the cheeks, of the gums, of the tongue, of the palate, or of the pharynx, followed by passage into the sublingual veins and distribution to the general venous circulation, thus short-circuiting the digestive tract and hepatic metabolism.

However, using this route is not obvious because setron molecules are all lipophilic and therefore virtually insoluble in the exclusively aqueous and hydrophilic buccal mucosal atmosphere.

Patent applications WO-2008/079295 and WO-2005/032520 describe per/sublingual formulations to be administered in the form of sprays. However, those compositions have characteristics that are unsatisfactory in terms of the administration accuracy, the absorption yields, and the bioavailability of the doses administered. Those are complex liquid formulations that include a combination of multiple ingredients intended to solubilize and stabilize ondansetron salts and to create a very specific viscosity state to distribute particles of particular size by spraying. During administration, distribution within the oral cavity remains diffuse and random, however, and immediately on reception of particles propelled by the spray, the particles are instantly mixed with the saliva produced mechanically by reflex in the oral cavity. This mixture is generally automatically swallowed by the patient before the active ingredient has had the opportunity to pass through the buccal mucosa to enter the venous circulation. The bioavailability curves given in patent application WO-2008/079295 show this loss of dose, the setron molecules administered by means of a spray by the method described being absorbed only partially via the buccal transmucosal route and mainly via the digestive tract. Only a very small fraction of the formulated active ingredient, never exceeding 20% (see WO-2008/079295, page 30, example 6) is therefore directly available via the transmucosal route, and efficacy remains very far from that obtained by the intravenous route.

There is therefore a need for a galenic formulation that is simple to produce and to use, less costly, readily available and not especially invasive, allowing administration of an immediately and completely bioavailable quantity of setrons so as to be able to treat very quickly and effectively major nausea and/or vomiting syndromes or spastic problems disabling the digestive tract.

The present invention addresses this need by proposing a highly specific galenic formulation, in the form of a solution, making it possible to guarantee transmucosal administration of at least one anti-nausea, anti-emetic, and/or digestive anti-spasmodic active ingredient from the setron family, consisting of:

at least one active ingredient from the setron family in base and/or salt form;

a hydroalcoholic solution consisting of water and ethanol, titrating at least 30° alcohol, in which said active principle is present in a stable and completely dissolved state; and

optionally, a pH corrector agent.

The pH of the formulation lies in the range from 5.0 to 9.0.

The invention also proposes a method of preparing this formulation and its use for the treatment or prevention of major nausea and/or vomiting syndromes and for treating or preventing disabling digestive tract spasm disorders.

Compared to existing formulations, the formulation of the invention has the advantage that is very simple to manufacture and to use and enables instantaneous and complete transmucosal passage of a setron-based therapeutic preparation, limiting both dilution by saliva and swallowing of the setron molecules, which molecules are delivered quasi-instantaneously to the vascular system for distribution of the entire dose to the receptor centers of the central nervous system. The dose of setrons administered is lower than that which needs to be introduced in existing formulations.

Other features and advantages emerge from the following description of the invention.

Thus a first aspect of the invention consists in a formulation for buccal transmucosal administration of at least one anti-nausea, anti-emetic, and/or digestive anti-spasmodic active ingredient from the setron family. This formulation is a solution having a pH in the range 5.0 to 9.0 and consisting of:

at least one active ingredient from the setron family in base and/or salt form;

a hydroalcoholic solution consisting of water and ethanol, titrating at least 30° alcohol; and

optionally, a pH corrector agent.

The active ingredient is present in a stable and completely dissolved state in the hydroalcoholic solution of less than 2 mL volume, to allow rapid absorption of said active ingredient via the mucosa of the oral cavity.

The expression “transmucosal route” refers to any passive passage of a lipophilic or amphiphilic molecule presented in a stable dissolved state through the mucosa of the tongue, under the tongue, of the gums, of the palate, of the cheeks, or any other mucosa of the oral cavity.

The expression “stable and completely dissolved state” refers to a solution state rendering the active ingredient in the molecular and weakly ionized state in its solution medium, this solution state preventing any possibility of inopportune recrystallization. This stable and completely dissolved state may be monitored immediately on use of the formulation of the invention by evaluation of the visual appearance of the solution obtained (measurement of its degree of limpidity) and then at the level of the filtration residues (appearance or non-appearance of crystals), and finally in the medium-term and long-term during stability tracking tests at various temperatures and relative humidities.

The expression “hydroalcoholic solution titrating X degrees alcohol” refers to a solution presenting a degree of alcohol equal to X, corresponding to the ratio between the volume of pure)(100° alcohol contained in the hydroalcoholic solution and the total volume of that solution. The degree of alcohol of the hydroalcoholic solution varies as a function of the degree of the alcohol used to form the solution and the water/alcohol ratio of the solution. For example, for 100° alcohol and a water/alcohol ratio of 50/50, the hydroalcoholic solution titrates 50° alcohol.

By pH corrector agent is meant any acid or base agent not degrading the physico-chemical characteristics of the active ingredient or ingredients.

The pH corrector agent is preferably chosen from carbonates and bicarbonates of sodium, monosodium or disodium phosphates, triethanolamine, sodium hydroxide (NaOH) and potassium hydroxide (KOH), and also hydrochloric, sulfuric, phosphoric, citric, malic, lactic, succinic, and/or butyric acid agents

The active ingredient from the setron family is present in base form and/or in salt form.

If the active ingredient is present in base form only, the formulation of the invention preferably contains an acid pH corrector agent.

If the active ingredient is present in salt form only, the formulation of the invention preferably contains a base pH corrector agent.

If the active ingredient is present in base form and in salt form, for example in succinate, chlorhydrate, or sulfate form, the distribution gradient between base and salt is determined extemporaneously as a function of the specific physico-chemical characteristics of each active ingredient and its salt, as is the dose, i.e. the concentration of the active ingredient relative to the volume of solution.

In a preferred embodiment, the active ingredient is present in base form. Setrons in base form, of lower molecular weight than setrons in salt form, dissolve and stabilize more easily in the formulation of the invention and have a greater aptitude for faster transmucosal passage.

The active ingredient may be chosen from ondansetron, tropisetron, granisetron, dolasetron, itasetron, alosetron, azasetron, benesetron, cliansetron, ramosetron, and zatosetron. The active ingredient is preferably ondansetron, granisetron, or tropisetron. The active ingredient is even more preferably ondansetron in the base form.

The formulation of the invention preferably takes the form of a hydroalcoholic solution containing 30% to 95% alcohol by volume and a water content in the range 5 to 70%. The formulation of the invention even more preferably takes the form of a hydroalcoholic solution containing 40% to 85% ethanol by volume and a water content in the range 15% to 60%.

The hydroalcoholic solution has a degree of alcohol of at least 30°, preferably in the range 30° to 70°, even more preferably in the range 40° to 70°, and ideally around 50°.

The hydroalcoholic solution is advantageously the only solvent used in the formulation of the invention.

Furthermore, the ethanol of the hydroalcoholic solution serves not only as a diluent, but also to promote accelerated transmucosal absorption, the speed of which increases as a function of the elevation of the degree of alcohol used. The degree of alcohol of the formulation must nevertheless not exceed 70° because a higher degree would be incompatible with a pharmaceutical composition for buccal application because of burns to the mucosa.

By way of example, the coefficient of dissolution of ondansetron in ethanol allows complete dissolution of said active ingredient at the rate of 2 milligrams (mg) of ondansetron per 0.75 milliliters (mL) of approximately 50° ethanol. This coefficient may be modulated as a function of the degree of alcohol and the water/ethanol ratio used.

The pH of the formulation of the invention is in the range 5.0 to 9.0, preferably in the range 5.5 to 7.5. These pH values are favorable to optimum absorption of the solution.

The formulation of the invention allows the active ingredient to pass passively through the buccal mucosa within 6 seconds of administration. This very short absorption time makes it possible to prevent any stagnation of the solution and the active ingredient in the buccal atmosphere, and to prevent their inopportune mixing with saliva liable to degrade them, which would introduce a break into the continuity and the stability of the dissolution of the active ingredient or ingredients. This short delay also makes it possible to prevent any reflex swallowing of the solution and the active ingredient that it contains.

The transmucosal passage of the active ingredient presented in the dissolved state of the invention to the external epithelial membrane, consisting of phospho-lipid structures that absorb passively by elective affinity the lipophilic molecules present in a stable and completely dissolved state is based on osmotic, or pulling, pressure towards the other side of said membrane, in which the concentration of dissolved active ingredient and the concentration of the alcohol solution concerned participate jointly. The activity and strength of the osmotic pressure increase with the degree of alcohol that serves as absorption promoter. In particular with ondansetron, according to the invention, an appropriate degree of alcohol is in the range 40° to 70°, preferably in the range 45° to 60°. This makes it possible to ensure simultaneously obtaining and setting the best coefficient of dissolution and of stabilization of ondansetron and promoting its transmucosal passage within 4 to 6 seconds. One particularly suitable embodiment corresponds to 0.75 mL of hydroalcoholic solution with approximately 50° alcohol per 2 mg or 4 mg of ondansetron.

The mucosa of the mouth have a very dense quasi-spongy array of micro-vessels, with the result that the molecules, either of the alcohol solvent or of the dissolved active ingredient, that pass through the lipophilic pores of the epithelial membrane are instantly captured by the micro-circulation of blood and collected toward the sublingual veins, and then the jugular veins to the heart. This phenomenon is accentuated by the presence of the alcohol, which causes vasodilation and increases the local micro-vascular flow rate of the mucosa.

Because of this locally raised circulatory flow rate, increased by the alcohol, there is therefore never equilibrium on respective opposite sides of the epithelial membrane: the concentration in the mouth always remains higher, until exhaustion of the mechanism for lack of molecules to absorb.

Thus, in distinct contrast to all other so-called “sublingual” forms, all of the alcohol and the active ingredient of the invention dissolved therein passes through the mucosa.

Use of the galenic formulation of the invention thus makes it possible to administer passively a dose of setrons that is absorbed immediately when deposited on the mucosa, and that is instantly distributed by the vascular route, with no delay in respect of its pharmacological action, and without suffering the destructive prior effects of digestive and hepatic passage. The galenic formulation of the invention thus enables tissue to absorb setron molecules immediately and completely, and this enables them to be distributed in the central circulation of the organism, generating by a rapid pharmacological response of the “flash” type.

For example, with a galenic formulation of the invention produced from 2 mg of ondansetron in base form dissolved in 0.75 mL of a 50° ethanol solution, it is possible to administer passively and virtually instantaneously a highly significant dose of ondansetron. This 2 mg dose corresponds to the theoretical maximum fraction available from a dose normally administered orally, i.e. in the range 40% to 50% at best of the dose usually administered orally. With the formulation of the invention, the dose administered by the local transmucosal route is completely bioavailable.

The hydroalcoholic solution of the invention, titrating at least 30° alcohol, also has the advantage of dissolving setron molecules even though they are lipophilic, which allows their spontaneous transmucosal absorption and protects the pharmaceutical formulation against microbiological contamination without having to introduce anti-microbial preservation agents.

Thus the hydroalcoholic solution of the invention is of four-fold efficacy:

it serves as the solvent for the active ingredient from the setron family, which are lipophilic molecules of low molecular weight;

it activates transmucosal passage of this dissolved active ingredient presented in the molecular state to the lipophilic membrane;

the degree of alcohol doubly increases the rate of mucosal absorption by the osmotic effect and by bringing about reflex micro-vascular vasodilation that accelerates the local micro-circulation flow rate; and

it is its own stabilizing agent, which avoids the use of conventional additives.

The present invention advantageously offers very simple production and very good galenic stability: the extremely simplified water/ethanol solution guarantees dissolution of the active ingredient and allows the excipients that are usually employed for conventional pharmaceutical preparations, including preservatives, to be omitted. The only optional additive is a pH corrector to adjust the pH of the solution to lie in the range 5.0 to 9.0.

The invention thus makes it possible to reduce manufacturing costs and also any risks of intolerance and of interaction between active ingredient and excipients.

Another advantage is that the delay in the pharmacodynamic action of the galenic formulation of the invention is very short, compared to the slow absorption of existing medications based on setrons that impose a delay in the range 45 minutes to 2 hours between taking the medication and the start of the anti-nausea, anti-emetic, or anti-spasmodic pharmacological action.

Quasi-instantaneous pharmacological delivery may enable a patient personally to administer a composition with an efficacy equivalent to the efficacy of a flash intravenous injection of setrons into the circulation, without the drawbacks linked to this type of administration, and in particular the risks of nasocomial infection.

This administration is much better in terms of simplicity and availability of non-traumatic administration but also in terms of unit and therapeutic cost compared to existing modes of administering setrons. The gain in terms of dose/effect ratio is at least 40% to 50%. With the formulation of the invention an at least 40% to 50% lower dose is used and a therapeutic effect is obtained without delay. The setron molecules encounter no significant obstacle to their instantaneous distribution via the carotid arteries to the target 5-HT3 receptors of the central nervous system, which they reach in a few seconds, and the base dose that needs to be administered is smaller, and comparable to the bioavailable dose needed for exercising the required pharmacological activity. The dose of active ingredient contained in the formulation of the invention is therefore lower than the doses conventionally administered. The dose is of course dependent on the setron being administered and on the required effect. It is preferably in the range 2 mg to 8 mg of active ingredient for hydroalcoholic solution volumes in the range 0.5 mL to 2 mL.

What is more, since the buccal mucosa have an extremely large total absorption area, increased by its creased villous tissue character, administering the galenic formulation of the invention is free from any risk of untimely swallowing and false routing. It allows extremely fast transmucosal passage that prevents any dissolving in saliva or swallowing of the active ingredient administered, with the advantage of not destabilizing the mucosa with various elements or excipients, as happens with some existing “sublingual” formulations in the form of sprays, slow-release pills, polymer membranes, or capsules. Moreover, the formulation of the invention is particularly suitable for patients suffering from major nausea or vomiting syndromes, because it completely avoids rejection of the ingested medication through vomiting.

Moreover, the effects of the alcohol are insignificant. For example, 0.75 mL of a 50° ethanol hydroalcoholic solution could only result in a alcohol blood level below 0.005 g per liter of blood, according to the official Widmark reference formula, i.e. one hundredth of the legal limit in France, which is set at 0.5 g per liter of blood. Moreover, the initial pulmonary passage of the alcohol solution should allow virtually complete elimination of the ethanol in the form of vapor extracted via the respiratory route and exhaled before the ethanol can be distributed in the organism. The alcohol vector is thus eliminated almost completely via the respiratory parenchyma.

A second aspect of the invention relates to a method of preparing the formulation.

A method of producing the particularly suitable galenic formulation of the invention comprises the following steps:

mixing alcohol and purified water and introducing into the mixture at least one active ingredient from the setron family;

stirring the preparation until a homogeneous suspension is obtained;

optionally, progressively introducing a pH corrector agent until the required pH in the range 5.0 to 9.0 is obtained;

continuing stirring until complete dissolution of the active ingredient;

adding water if necessary to make up to the required volume; and

filtering.

In a preferred implementation the method comprises the following steps:

mixing alcohol and purified water and introducing into the mixture ondansetrol in base and/or salt form;

stirring the preparation, preferably for 10 to 60 minutes, until a homogeneous suspension is obtained;

optionally, progressively introducing a pH corrector agent until the required pH in the range 5.0 to 9.0 is obtained;

continuing stirring, preferably for 5 to 30 minutes, until complete dissolution of the active ingredient;

adding water if necessary to make up to the required volume; and

filtering.

In a first variant, the method of the invention comprises the following steps:

mixing ethanol and water and introducing into the mixture an active ingredient from the setron family in base form;

stirring the preparation, preferably for 10 to 60 minutes, until a homogeneous suspension is obtained;

optionally, progressively introducing an acidic pH corrector agent until a pH in the range 5.0 to 7.0, preferably close to 6.0, is obtained;

continuing stirring, preferably for 5 to 30 minutes, until complete dissolution of the active ingredient;

adding water if necessary to make up to the required volume; and

filtering using a 5 micrometer (pm) filter and dispensing the preparation into single-dose bottles.

In a second variant, the method of the invention comprises the following steps:

mixing ethanol and water and introducing into the mixture an active ingredient from the setron family in salt form;

stirring the preparation, preferably for 10 to 60 minutes, until a homogeneous suspension is obtained;

optionally, progressively introducing a basic pH corrector agent until a pH in the range 6.0 to 8.0, preferably close to 7.0, is obtained;

continuing stirring, preferably for 5 to 30 minutes, until complete dissolution of the active ingredient;

adding water if necessary to make up to the required volume; and

filtering using a 5 μm filter and dispensing the preparation into single-dose bottles.

In another variant, the method of the invention comprises the following steps:

mixing ethanol and water and introducing into the mixture an active ingredient from the setron family in salt form;

stirring the preparation, preferably for 10 to 60 minutes, until a homogeneous suspension and complete dissolution of the active ingredient are obtained;

adding water if necessary to make up to the required volume; and

filtering using a 5 μm filter and dispensing the preparation into single-dose bottles.

The present invention may be used for the instantaneous systemic administration of reduced and effective doses of setrons, in particular ondansetron.

The formulation of the present invention may in particular be used to produce a medication for the treatment and/or prevention of major nausea and/or vomiting syndromes, in particular linked to cancer treatment. Such a medication has a therapeutic anti-emetic activity within a very short time and at doses very greatly reduced compared to the conventional doses.

The formulation of the present invention may also be used to produce a medication for the treatment and/or prevention of digestive spasms.

The formulation of the invention, corresponding to a very small liquid volume, is very easy to administer. A patient may easily place it in the mouth in direct contact with a precise small mucosal area of the mouth, the gums, or under the tongue.

According to a final aspect of the invention, the formulation requires specific industrial packaging in order to allow it to be used safely, simply and ergonomically and in order to prevent the active ingredient from being degraded by contact with air.

One particular embodiment consists in using opaque glass or flexible metal-plastic or plastic packaging, preferably of small size, filled in an inert atmosphere such as nitrogen, in order to protect the stability of the composition and in order to provide impermeability to oxygen and to radiation. These forms of packaging guarantee dissolution and long-term stability of the dissolved active ingredients of the invention in hydroalcoholic solution.

These forms of packaging preferably include a cannula allowing precise deposition of the solution of the invention in contact with an appropriate area of the mucosa.

For comfortable use by the patient, for easy transportation, dedicated sealed packages may preferably be used for packaging. Even more preferably, the galenic formulation of the invention is packaged in 0.5 mL to 2 mL single-dose packages suitable for providing an adequate dose of active ingredient.

This packaging is advantageously easy to transport and allows easy use of the galenic formulation at any time of day.

Examples of setron formulations of the invention may be mentioned, with a volume of 0.75 mL or 1.00 mL, at approximately 50° alcohol, particularly suited to producing effective action at the level of the central nervous system with a delay of only a few minutes:

### Formulation 1

### 2 mg Ondansetron, 0.75 mL of 50° Alcohol

ondansetron in the base form (active ingredient): 2.0 mg

95° ethyl alcohol (diluent and absorption promoter): 0.375 mL

purified water (diluent): qsp 0.75 mL

hydrochloric acid (pH corrector): qsp pH 6.0

This first formulation example may be obtained using the method described below for a batch of 1000 doses, i.e. 0.75 liters (L).

Into a stainless steel tank introduce 0.375 L of 95% V/V ethanol and 0.150 L of purified water.

Introduce into the hydroalcoholic solution 2 grams (g) of ondansetron in the base form.

Using a helical stirrer, stir the preparation for 20 to 40 minutes until a homogeneous suspension is obtained.

Then progressively add hydrochloric acid until a pH close to 6 is obtained (plus or minus 1).

Continue stirring until complete dissolution of the ondansetron.

Make up with purified water to obtain a solution of 0.75 L volume and stir the preparation for 10 to 30 minutes to ensure its homogeneity.

Filter the preparation using a polypropylene or like filter of 5 μm porosity and dispense the preparation into 0.75 mL single-dose bottles.

### Formulation 2

### 4 mg Ondansetron, 0.75 mL of 50° Alcohol

ondansetron in base form (active ingredient): 4.0 mg

95° ethyl alcohol (diluent and absorption promoter): 0.375 mL

purified water (diluent): qsp 0.75 mL

hydrochloric acid (pH corrector): qsp pH 6.0

This second formulation example may be obtained using the method described below for a batch of 1000 doses, i.e. 0.75 liter.

Into a stainless steel tank introduce 0.375 L of 95% V/V ethanol and 0.150 L of purified water.

Introduce into the hydroalcoholic solution 4 g of ondansetron in base form.

Using a helical stirrer, stir the preparation for 20 to 40 minutes until a homogeneous suspension is obtained.

Then progressively add hydrochloric acid until a pH close to 6 is obtained (plus or minus 1).

Continue stirring until complete dissolution of the ondansetron.

Make up with purified water to obtain a solution of 0.75 L volume and stir the preparation for 10 to 30 minutes to ensure its homogeneity.

Filter the preparation using a polypropylene or like filter of 5 μm porosity and dispense the preparation into 0.75 mL single-dose bottles.

### Formulation 3

### 4 mg Ondansetron, 1.0 mL of 50° Alcohol

ondansetron in base form (active ingredient): 2.0 mg

HCl ondansetron (active ingredient): 2.0 mg

95° ethyl alcohol (diluent and absorption promoter): 0.5 mL

purified water (diluent): qsp 1.0 mL

This formulation example may be obtained using the method described below for a batch of 1000 doses, i.e. 1 liter.

Into a stainless steel tank introduce 0.5 liter of 95% V/V ethanol and 0.5 L of purified water.

Introduce into the hydroalcoholic solution 2 g of ondansetron in base form and 2 g of HCl ondansetron.

Using a helical stirrer, stir the preparation for 20 to 40 minutes until a homogeneous suspension and complete dissolution of the ondansetron are obtained.

Filter the preparation using a polypropylene or like filter of 5 μm porosity and dispense the preparation into 1.0 mL single-dose bottles.

### Formulation 4

### 3 mg Granisetron, 1.0 mL of 50° Alcohol

HCl Granisetron (active ingredient): 3.0 mg

95° ethyl alcohol (diluent and absorption promoter): 0.5 mL

purified water (diluent): qsp 1.0 mL

NaOH (pH corrector) : qsp pH 7.5

This formulation example may be obtained using the method described below for a batch of 1000 doses, i.e. 1.0 liter.

Into a stainless steel tank introduce 0.500 L of 95% V/V ethanol and 0.350 L of purified water.

Introduce into the hydroalcoholic solution 3 g of HCl Granisetron.

Using a helical stirrer, stir the preparation for 20 to 40 minutes until a homogeneous suspension is obtained.

Then progressively add hydrochloric acid until a pH close to 7.5 is obtained (plus or minus 1).

Continue stirring until complete dissolution.

Make up with purified water to obtain a solution of 1.0 liter volume and stir the preparation for 10 to 30 minutes to ensure its homogeneity.

Filter the preparation using a 5 μm polypropylene or like filter and dispense the preparation into 1.0 mL single-dose bottles.

Of course, the invention is obviously not limited to the examples shown and described above, but on the contrary covers all variants thereof.

1. A formulation for the buccal transmucosal administration of at least one anti-nausea, anti-emetic and/or digestive anti-spasmodic active ingredient from the setron family, the formulation being characterized in that it comprises a solution having a pH in the range 5.0 to 9.0 and comprising:
at least one active ingredient from the setron family in base and/or salt form;
a hydroalcoholic solution comprising water and ethanol, titrating at least 30° alcohol, in which said active principle is present in a stable and completely dissolved state; and
optionally, a pH corrector agent.

2. A formulation according to claim 1, characterized in that the pH corrector agent is chosen from carbonates and bicarbonates of sodium, monosodium or disodium phosphate, triethanolamine, sodium hydroxide, potassium hydroxide and/or from, sulfuric, succinic, butyric, phosphoric, citric, malic, and/or lactic acid agents.

3. A formulation according to claim 1, characterized in that the active ingredient is in base form and the pH corrector agent is an acid agent.

4. A formulation according to claim 1, characterized in that the active ingredient is in salt form and the pH corrector agent is a base agent.

5. A formulation according to claim 1, characterized in that the active ingredient is present in base form and in succinate, chlorhydrate, or sulfate form.

6. A formulation according to claim 1, characterized in that the pH is in the range 5.5 to 7.5.

7. A formulation according to claim 1, characterized in that the hydroalcoholic solution titrates in the range 30° to 70° alcohol.

8. A formulation according to claim 1, characterized in that the hydroalcoholic solution contains in the range 30% to 95% alcohol and 5% to 70% water by volume.

9. A formulation according to claim 1, characterized in that the active ingredient is ondansetron, granisetron, tropisetron, dolasetron, itasetron, azasetron, benesetron, cliansetron, ramosetron or zatosetron.

10. A formulation according to claim 1, characterized in that it contains in the range 2 mg to 8 mg of active ingredient for volumes of hydroalcoholic solution in the range 0.5 mL to 2 mL.

11. A method of preparing a formulation according to claim 1, characterized in that it comprises the following steps:
mixing alcohol and purified water and introducing into the mixture at least one active ingredient from the setron family;
stirring the preparation until a homogeneous suspension is obtained;
optionally, progressively introducing a pH corrector agent until the required pH in the range 5.0 to 9.0 is obtained;
continuing stirring until complete dissolution of the active ingredient;
adding water if necessary to make up to the required volume; and
filtering.

12. A method according to claim 11 of preparing a formulation, characterized in that it comprises the following steps:
mixing ethanol and purified water and introducing into the mixture ondansetron in base and/or salt form;
stirring the preparation, preferably for 10 to 60 minutes, until a homogeneous suspension is obtained;
optionally, progressively introducing a pH corrector agent until the required pH in the range 5.0 to 8.0 is obtained;
continuing stirring, preferably for 5 to 30 minutes, until complete dissolution of the active ingredient;
adding water if necessary to make up to the required volume; and
filtering.

13. Use of the formulation according to claim 1 for the production of a medication intended to treat by buccal transmucosal administration major nausea and/or vomiting syndromes.

14. Use of the formulation according to claim 1 for the production of a medication intended to treat and/or prevent digestive spasms by buccal transmucosal administration.
